(19) United States
(12) Patent Application Publication
(10) Pub. No.: US 2023/0083584 A1
(43) Pub. Date: Mar. 16, 2023

(54) ELECTROSURGICAL VESSEL SEALING DEVICE CONTROLLER

(71) Applicant: Conmed Corporation, Largo, FL (US)

(72) Inventors: Roberto Del Cid, Denver, CO (US); Brian Stuebe, Golden, CO (US)

(73) Assignee: Conmed Corporation, Largo, FL (US)

(21) Appl. No.: 17/798,207

(22) PCT Filed: Feb. 5, 2021
(86) PCT No.: PCT/US2021/016798

§ 371 Date: Aug. 8, 2022

Related U.S. Application Data
(60) Provisional application No. 62/970,816, filed on Feb. 6, 2020.

(51) Int. Cl. A61B 18/14; A61B 18/12

(52) U.S. Cl. CPC A61B 18/1445; A61B 18/1206; A61B 2018/0063

(57) ABSTRACT

A power delivery approach for delivering power to an electrosurgical vessel sealer when the jaws of the sealer surround tissue to be desiccated. Power delivery commences at a starting point that is at least 40 Joules and then decreases over a first predetermined period of time to a predetermined minimum power level to provide approximately 15 Joules in total. When the predetermined minimum power level is reached, power is then continuously increased over a second predetermined period of time to fully desiccate the tissue. Power delivery is terminated prior to over-desiccation of the tissue.

### CROSS-REFERENCE TO RELATED APPLICATIONS

The present application claims priority to U.S. Provisional No. 62/970816, filed on Feb. 6, 2020, which is hereby incorporated by reference in its entirety.

### BACKGROUND OF THE INVENTION

### 1. Field of the Invention

The present invention relates surgical instruments and, more specifically, to an electrosurgical vessel sealing device controller that can produce repeatable seals in blood vessels and other tissues.

### 2. Description of the Related Art

Electrosurgical vessel sealers are surgical instruments that are used for the occlusion of blood vessels and halting of bleeding during surgical procedures. The electrodes of the vessel sealer are carried by a pair of opposing jaws and interconnected to an electrosurgical generator that can selectively supply radiofrequency (RF) energy to the electrodes. A user may close the jaws around a vessel to be sealed by squeezing a lever associated with a handle assembly. The vessel may then be sealed by supplying the RF energy to the clamped vessel.

Electrical power control of the vessel sealer is controlled by the electrosurgical generators. Conventional approaches to power control involve the application of power according to a predetermined power curve where power is variably applied to have a particular impact on the tissue to be sealed. Due to the variability of the tissue to be treated, however, these approaches often lack consistent results. Accordingly, there is a need in the art for an electrosurgical vessel sealing device controller that is configured to produce repeatable conditions in the tissue to be treated for consistent results.

### BRIEF SUMMARY OF THE INVENTION

The present invention provides an electrosurgical vessel sealing device controller that is configured to provide energy according to a scheme that conditions the tissue to be treated to be homogeneous by pre-polymerizing collagen and then allows rehydration of the tissue prior to the main sealing cycle. More specifically, the present invention includes an electrosurgical system having a vessel sealer having a pair of jaws and an electrosurgical generator coupled to the pair of jaws of the vessel sealer and having a controller configured to output radiofrequency energy to the vessel sealer. The controller is configured to output sufficient radiofrequency energy in a first stage at a first power level until a first stopping point, to interrupt the output of sufficient radiofrequency energy in a second stage for a predetermined period of time, and to output sufficient radiofrequency energy in third stage at a second power level to cause sealing of the tissue. The stopping point is selected to elevate the temperature of any tissue positioned in the jaws without reaching a full polymerization temperature where any collagen and elastic in the tissue will fully polymerize. The stopping point may be selected according to the tissue achieving a minimum impedance for a minimum amount of time, the tissue reaching a predetermined impedance measured after a fixed amount of time, a fixed amount of time, and a rate of change of impedance of the tissue exceeding a threshold. The predetermined period of time of the second stage may comprise a fixed duration. The predetermined period of time of the second stage may comprise a variable duration. The variable duration may be determined based on the amount of time the tissue required to reach the minimum impedance of the first stage or the amount of time for the rate of change of impedance to exceed the threshold in the first stage. The third stage may comprise the output of power at the second power level until the tissue reaches an end point. The third stage may comprise the output of power at the second power level until the tissue reaches an intermediate impedance at a fixed time. The third stage may comprise the output of power at the second power level that is proportional to the time taken to exceed the impedance rate of change threshold from the first stage.

The present invention also includes a method of controlling the power output from an electrosurgical generator to a vessel sealer having a pair of jaws. The method includes the steps of providing the vessel sealer having the pair of jaws, coupling the electrosurgical generator to the pair of jaws of the vessel sealer, and powering the electrosurgical generator to output radiofrequency energy to the vessel sealer in a first stage at a first power level until a first stopping point, to interrupt the output of sufficient radiofrequency energy in a second stage for a predetermined period of time, and to output sufficient radiofrequency energy in third stage at a second power level to cause sealing of the tissue. The stopping point may be selected to elevate the temperature of any tissue positioned in the jaws without reaching a full polymerization temperature where any collagen and elastic in the tissue will fully polymerize. The stopping point may be selected based on the tissue achieving a minimum impedance for a minimum amount of time, the tissue reaching a predetermined impedance measured after a fixed amount of time, a fixed amount of time, and a rate of change of impedance of the tissue exceeding a threshold. The predetermined period of time of the second stage may comprise a fixed duration. The predetermined period of time of the second stage may comprise a variable duration that is determined based on the amount of time the tissue required to reach the minimum impedance of the first stage or the amount of time for the rate of change of impedance to exceed the threshold in the first stage.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

The present invention will be more fully understood and appreciated by reading the following Detailed Description in conjunction with the accompanying drawings, in which:

FIG. 1 is a perspective view of an electrosurgical system according to the present invention

FIG. 2 is a schematic of an electrosurgical generator for an electrosurgical system that may be configured to deliver power according to the present invention;

FIG. 3 is a power delivery curve according to a first embodiment the present invention;

FIG. 4 is a power delivery curve according to another embodiment of a power control algorithm of the present invention;

FIG. 5 is a power delivery curve according to a further embodiment of a power control algorithm of the present invention; and

FIG. 6 is a power delivery curve according to an additional embodiment of a power control algorithm of the present invention.

### DETAILED DESCRIPTION OF THE INVENTION

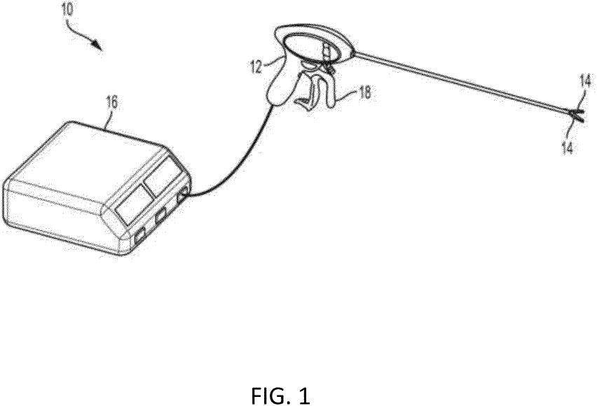

Referring to the figures, wherein like numeral refer to like parts throughout, there is seen in FIG. 1 an electrosurgical system 10 comprising a vessel sealer 12 having a pair of conductive opposing jaws 14 that are interconnected to an electrosurgical generator 16 that can supply RF energy to electrodes of jaws 14 for the desiccation of a blood vessel trapped between jaw 14. The dimensions of jaws 14 and the type of RF energy supplied will produce desiccation of the blood vessel in a region of a particular width as determined by the thermal spread of the energy being supplied to the blood vessel. As is known in the art, jaws 14 are pivotally mounted to vessel sealer 12 for movement between an open position and a closed position in response to a user operating a lever 18 extending from the main body 20 of sealer 12.

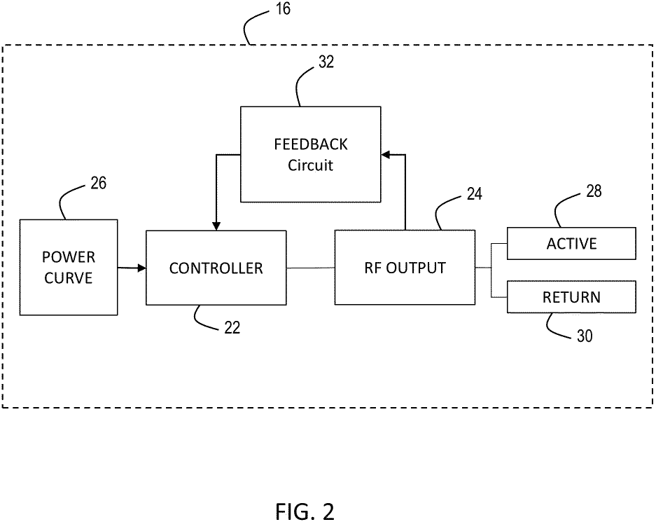

Referring to FIG. 2, electrosurgical generator 16 comprises the electronics and circuity response for precisely delivering RF energy to jaws 14 according to the present invention. The components, connections, sensing elements, electrical circuits, power source, user controls, and programmable control elements of electrosurgical generator 16 are known in the art and only discussed herein with respect to the specific aspects of electrosurgical generator 16 that are configured for implementation of the present invention. More specifically, electrosurgical generator 16 comprises a controller 22 that is programmed to execute substantially all of the feedback and regulation functionality of electrosurgical generator 16 and, more specifically, output radiofrequency energy from an RF output 24 according to one or more predefined power delivery curves according to the present invention that are stored in a power curve module 26. RF output 24 is coupled to an active path 28 and a return 30 of electrosurgical generator 16 that may be coupled to an electrosurgical instrument, such as vessel sealer 12, to deliver radiofrequency according to one or more of the power delivery curves to jaws 14 and thus desiccation of any tissue trapped within jaws 14. Electrosurgical generator 16 includes feedback circuit 32 so that controller 22 can monitor the output of radiofrequency energy from RF output 24. As is known in the art, feedback information provided by feedback circuit 32 can be used by controller 22 to control, regulate, and adjust the delivery of radiofrequency according to one or more of the power delivery curves, and to monitor the amount of power delivered to vessel sealer 12 over time. Most specifically, controller 22 of electrosurgical generator 16 is programmed to output power to jaws 14 according to a three phase approach that has a first stage where energy is provided in a manner to condition the blood vessel or other tissue, a second stage where the vessel is allowed to re-hydrate, and a third stage where sufficient energy to seal the vessel is provided. approach implemented by controller 22 commences with the delivery of an initial conditioning pulse of energy according to a first power level according to a first load curve.

The first stage implemented by controller 22 comprises an initial conditioning pulse that is configured to elevate the temperature of the tissue to a level above body temperature, but below the full polymerization temperature of the collagen and elastin in the tissue. Polymerization generally starts around 70° C. to 80° C. but can be more readily identified by the change in impedance, which is primarily a factor of the moisture that is being affected by the energy delivery. The amount of moisture can relate to temperature rise as the moisture will evaporate around about 100° C. and there will not be a sharp rise in temperature past that point until the tissue starts to dry out. There is a relationship between the power and time to keep the temperature low and is a function of energy delivery. If the power is higher, the time duration of pre-conditioning phase has to be shorter. For example, pre-conditioning can occur at 40 Watts for 250 milliseconds or 20 Watts for 400 milliseconds (as compared to a sealing stage at 40 W that is greater than 750 milliseconds and provides full polymerization). It should be recognized by those of skill in the art that these amounts are dependent on many factors, such as vessel size. A small vein, for example, can be fully polymerized at 750 ms. A larger artery might take 2-3 seconds. The dimensions of the jaw, jaw pressure and other factors may also be relevant. Nevertheless, in any circumstances, the first stage is unlikely to be greater than 750 ms.

The elevation of temperature while remaining below the polymerization temperature is accomplished by stopping the delivery of energy at one of a number of alternative stopping points before the temperature becomes to high. One acceptable stopping point may comprise the tissue reaching a minimum impedance for a minimum amount of time. The stopping point may instead comprise a predetermined impedance that is measured after a fixed amount of time has elapsed. The stopping point may also comprise a fixed amount of time having elapsed. The stopping point may additionally comprise the rate of impedance change exceeding a predetermined threshold.

After completion of the first stage, controller 22 is configured to implement a second stage where the delivery of energy is interrupted for a predetermined period of time to allow for the rehydration of the tissue. The predetermined period of time may comprise a fixed duration or a variable duration. The variable duration may be determined based on the time that the tissue took to reach the predetermined minimum impedance of the conditioning pulse in the first stage, the particular type of handpiece being used, or the amount of time for the rate of impedance change to exceed the predetermined threshold in the first stage.

After completion of the second stage, controller 22 is configured to implement a sealing cycle. For the sealing cycle, power is applied according to a conventional load curve to a second power level that is greater than the first power level and energy is delivered until the tissue reaches a desired end point. Second power level may be between 40W and 50W. Alternatively, power may be applied according to a conventional load curve to a second power level that is greater than the first power level until the tissue reaches an intermediate impedance at a fixed time. The energy delivery may then be decreased (“stepped down”) to a third power level, such as 30W, hat is lower than the second power level and applied according to a third load curve. Energy delivery is then maintained until the tissue reaches a desired end point. Finally, power may be applied at a power level that is proportional to the time taken to exceed the impedance rate of change threshold from the first stage, such as between 30W and 100W.

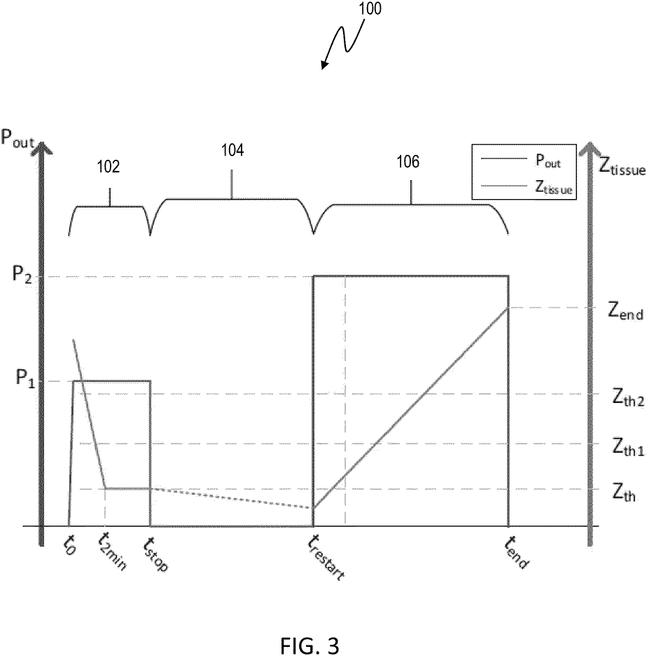

Referring to FIG. 3, controller 22 can implement a three phase approach 100 having a first conditioning stage 102 and P₁, followed by a re-hydration stage 104 and finally a sealing stage 106 at P₂. As seen in FIG. 3, the tissue impedance decreases over time, during first stage 102, from an initial level at the start of energy deliver, t₀, to a minimum level at t₂ₘᵢₙ and stays relatively constant until energy delivery is stopped at the end of first stage 102, tₛₜₒₚ. t₂ₘᵢₙ can occur at around 100 ms with 40 W of power delivery. The end of stage 102, tₛₜₒₚ, can occur after or a fixed time or after the measured tissue impedance is at or below impedance threshold Zₜₕ for a minimum amount of time or the measured tissue impedance is at or below the threshold Zₜₕ after a fixed amount of time. tₛₜₒₚ is likely to be at about 250 ms to 1.25 s depending on the tissue and the triggering event, such as impedance. The tissue impedance in rehydration stage 104 stays fairly constant or even decreases a bit until energy is applied again at sealing stage 106. tₛₜₒₚ to tᵣₑₛₜₐᵣₜ of rehydration state 104 would likely not exceed 250 ms, and will probably be closer to 50 ms. Sealing stage 106 is performed at power level P₂, which is greater than power level P₁. During sealing stage 106, the tissue impedance increases until the stopping point of stage 106. P₁ may be 25 W and be delivered for 250 ms to 300 ms (t₀ to tₛₜₒₚ). P₂ may be 40 W to 60 W and be delivered for 750 ms up to 4 seconds depending on the particular tissue to be treated and device conditions (tᵣₑₛₜₐᵣₜ to t_{end}). A desired end impedance, Z_{end}, may be used to determine when to stop at tend.

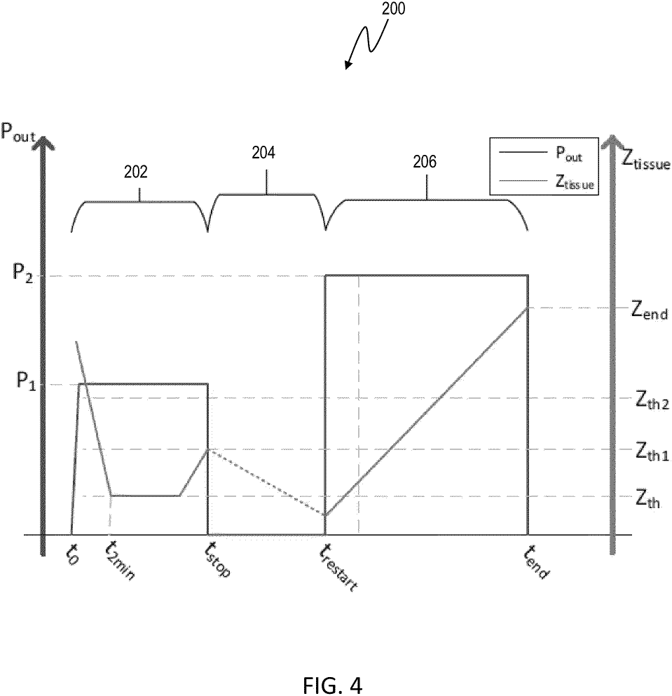

Referring to FIG. 4, controller 22 can implement a three-phase approach 200 having a first conditioning stage 202, followed by a re-hydration stage 204 and finally a sealing stage 206. As seen in FIG. 4, tissue impedance during power delivery of first stage 202 decreases over time from an initial level at t₀ to a minimum level at t₂ₘᵢₙ. The tissue impedance stays at a minimum for a period of 202 as the tissue desiccates. Stage 202 ends at tₛₜₒₚ when the controller 22 detects a sharp rise in the tissue impedance. Sealing stage 206 is performed at power level P₂, which is greater than power level P₁ of stage 202. During sealing stage 206, the tissue impedance increases until the stopping point of stage 206. The timing and power levels of the example seen in FIG. 4 are comparable to those of FIG. 3, with just the variations described herein.

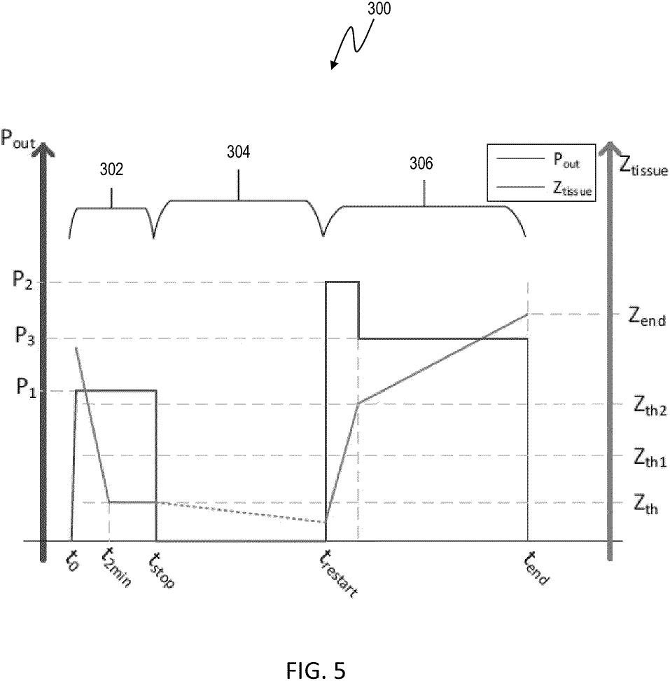

Referring to FIG. 5, controller 22 can implement a three-phase approach 300 having a first conditioning stage 302, followed by a re-hydration stage 304 and finally a sealing stage 306. As seen in FIG. 5, the tissue impedance decreases over time, during first stage 302, from an initial level at the start of energy deliver, t₀, to a minimum level at t₂ₘᵢₙ and stays relatively constant until energy delivery is stopped at the end of first stage 302, tₛₜₒₚ. The end of stage 102, tₛₜₒₚ, can occur after or a fixed time or after the measured tissue impedance is at or below impedance threshold Zₜₕ for a minimum amount of time or the measured tissue impedance is at or below the threshold Zₜₕ after a fixed amount of time. The tissue impedance in rehydration stage 304 stays fairly constant or even decreases a bit until energy is applied again at sealing stage 306. Sealing stage 306 comprises a first power delivery of power P2, which is greater than P1, at tᵣₑₛₜₐᵣₜ. At time t_{decision} the tissue impedance is measured and compared to impedance thresholds. If measured tissue impedance is greater than or equal to Zₜₕ₂, the power delivery in stage 306 will be reduced to P3 which is greater than P1 but less than P2. If the measured tissue impedance is less than Zₜₕ₂ the power delivery of stage 306 will remain at P2. Stage 306 will continue until stopping point t_{end}. The stopping point t_{end} occurs when the measured tissue impedance meets or exceeds impedance threshold Z_{end}. The timing and power levels of the example seen in FIG. 5 are comparable to those of FIG. 3, with just the variations described herein.

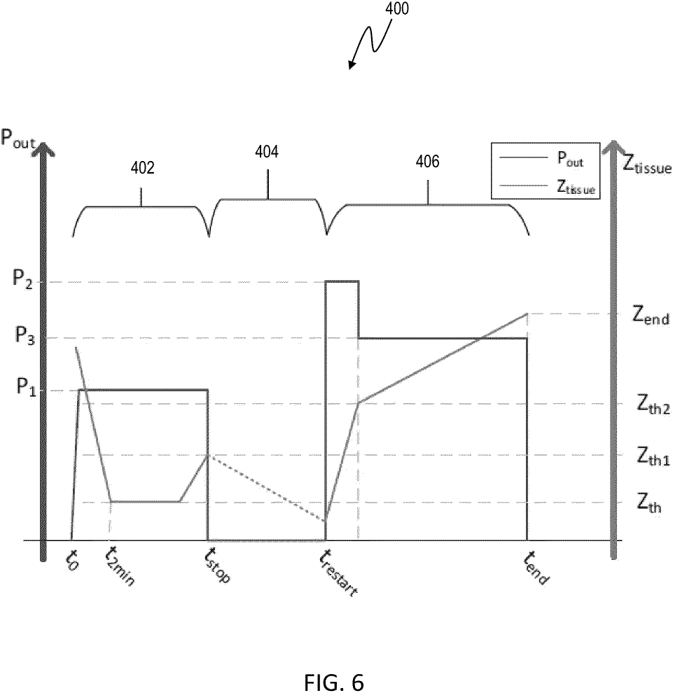

Referring to FIG. 6, controller 22 can implement a three-phase approach 400 having a first conditioning stage 402, followed by a re-hydration stage 404 and finally a sealing stage 406. As seen in FIG. 6, tissue impedance during power delivery of first stage 402 decreases over time from an initial level at t₀ to a minimum level at t₂ₘᵢₙ. The tissue impedance stays at a minimum for a period of 402 as the tissue desiccates Stage 402 ends at tₛₜₒₚ when the controller 22 detects a sharp rise in the tissue impedance. Sealing stage 406 comprises a first power delivery of power P2, which is greater than P1, at tᵣₑₛₜₐᵣₜ. At time t_{decision} the tissue impedance is measured and compared to impedance thresholds. If measured tissue impedance is greater than or equal to Zₜₕ₂, the power delivery in stage 306 will be reduced to P3 which is greater than P1 but less than P2. If the measured tissue impedance is less than Zₜₕ₂ the power delivery of stage 306 will remain at P2. Stage 306 will continue until stopping point t_{end}. The stopping point t_{end} occurs when the measured tissue impedance meets or exceeds impedance threshold Z_{end}. The timing and power levels of the example seen in FIG. 5 are comparable to those of FIG. 3, with just the variations described herein.

As described above, the present invention may be a system, a method, and/or a computer program associated therewith and is described herein with reference to flowcharts and block diagrams of methods and systems. The flowchart and block diagrams illustrate the architecture, functionality, and operation of possible implementations of systems, methods, and computer programs of the present invention. It should be understood that each block of the flowcharts and block diagrams can be implemented by computer readable program instructions in software, firmware, or dedicated analog or digital circuits. These computer readable program instructions may be implemented on the processor of a general purpose computer, a special purpose computer, or other programmable data processing apparatus to produce a machine that implements a part or all of any of the blocks in the flowcharts and block diagrams. Each block in the flowchart or block diagrams may represent a module, segment, or portion of instructions, which comprises one or more executable instructions for implementing the specified logical functions. It should also be noted that each block of the block diagrams and flowchart illustrations, or combinations of blocks in the block diagrams and flowcharts, can be implemented by special purpose hardware-based systems that perform the specified functions or acts or carry out combinations of special purpose hardware and computer instructions.

What is claimed is:

1. An electrosurgical system, comprising:
a vessel sealer having a pair of jaws; and
an electrosurgical generator coupled to the pair of jaws of the vessel sealer and having a controller configured to output radiofrequency energy to the pair of jaws of the vessel sealer;
wherein the controller is configured to output radiofrequency energy in a first stage at a first power level until a first stopping point, to interrupt the output of sufficient radiofrequency energy in a second stage for a predetermined period of time, and to output sufficient radiofrequency energy in third stage at a second power level to cause sealing of the tissue.

2. The electrosurgical system of claim 1, wherein the first stopping point is selected to elevate the temperature of any tissue positioned in the pair of jaws without reaching a temperature where any collagen and elastic in the tissue will fully polymerize.

3. The electrosurgical system of claim 2, wherein the first stopping point is selected from the group consisting of the tissue achieving a minimum impedance for a minimum amount of time, the tissue reaching a predetermined impedance measured after a fixed amount of time, a fixed amount of time, and a rate of change of impedance of the tissue exceeding a threshold.

4. The electrosurgical system of claim 3, wherein the predetermined period of time of the second stage comprises a fixed duration.

5. The electrosurgical system of claim 3, wherein the predetermined period of time of the second stage comprises a variable duration.

6. The electrosurgical system of claim 5, wherein the variable duration is determined based on the amount of time the tissue required to reach the minimum impedance of the first stage or the amount of time for the rate of change of impedance to exceed the threshold in the first stage.

7. The electrosurgical system of claim 3, wherein the third stage comprises the output of power at the second power level until the tissue reaches an end point.

8. The electrosurgical system of claim 3, wherein the third stage comprises the output of power at the second power level until a fixed time at which the output power may or may not switch to a third power level determined by the comparison of the impedance to a fixed threshold value.

9. The electrosurgical system of claim 3, wherein the third stage comprises the output of power at the second power level that is proportional to the time taken to exceed the impedance rate of change threshold from the first stage.

10. A method of controlling an amount of power output from an electrosurgical generator to a vessel sealer having a pair of jaws, comprising:
providing a vessel sealer having a pair of jaws;
coupling the electrosurgical generator to the pair of jaws of the vessel sealer; and
powering the electrosurgical generator to output radiofrequency energy to the vessel sealer in a first stage at a first power level until a first stopping point, to interrupt the output of radiofrequency energy in a second stage for a predetermined period of time, and to output sufficient radiofrequency energy in a third stage at a second power level to cause sealing of any tissue trapped within the pair of jaws.

11. The method of claim 10, wherein the first stopping point is selected to elevate the temperature of any tissue positioned in the pair of jaws without reaching a full polymerization temperature where any collagen and elastic in the tissue will fully polymerize.

12. The method of claim 11, wherein the first stopping point is selected from the group consisting of the tissue achieving a minimum impedance for a minimum amount of time, the tissue reaching a predetermined impedance measured after a fixed amount of time, a fixed amount of time, and a rate of change of impedance of the tissue exceeding a threshold.

13. The method of claim 12, wherein the predetermined period of time of the second stage comprises a fixed duration.

14. The method of claim 11, wherein the predetermined period of time of the second stage comprises a variable duration that is determined based on the amount of time the tissue required to reach the minimum impedance of the first stage or the amount of time for the rate of change of impedance to exceed the threshold in the first stage.
